(19) United States
(12) Patent Application Publication
(10) Pub. No.: US 2022/0160892 A1
(43) Pub. Date: May 26, 2022

(54) SINGLE NIR IRRADIATION TRIGGERED UPCONVERSION NANO SYSTEM FOR SYNERGISTIC PHOTODYNAMIC AND PHOTOTHERMAL CANCER THERAPY

(71) Applicant: Board of Regents, The University of Texas System, Austin, TX (US)

(72) Inventors: XiuJun Li, El Paso, TX (US); Luis A. Echegoyen, El Paso, TX (US); Lei Ma, El Paso, TX (US)

(21) Appl. No.: 17/456,534

(22) Filed: Nov. 24, 2021

Related U.S. Application Data
(60) Provisional application No. 63/117,896, filed on Nov. 24, 2020.

(51) Int. Cl. A61K 47/69; A61K 31/40; A61K 41/00; A61K 47/02

(52) U.S. Cl. CPC A61K 47/6949; A61K 31/40; B82Y 5/00; A61K 47/02; A61K 41/0057

(57) ABSTRACT

A composition includes a photon upconversion nanoparticle coupled to a photosensitizer nanoparticle that can absorb light and convert tissue oxygen into reactive oxygen species. A low temperature hydrothermal method of making photon upconversion nanoparticles includes dispersing Yb(NO₃)₃, Y(NO₃)₃, and Er(NO₃)₃ in water to prepare a mixture; adding an ethylenediaminetetraacetic acid and NaF with sonication to make a solution; adjusting a pH of the solution to approximately 3.5 using HNO₃ and NaOH; treating the solution hydrothermally at approximately 130° C. for approximately 4 hours; quenching to approximately 20° C.; collecting and washing the photon upconversion nanoparticles. A near infrared triggered photon upconversion method for synergistic photodynamic and photothermal cancer therapy includes administering a nanocomposite comprising a photon upconversion nanoparticle coupled to a photosensitizer nanoparticle that can absorb light and convert tissue oxygen into reactive oxygen species; and exposing the mammal and the nanocomposite to a near infrared source of actinic radiation.

### CROSS REFERENCE TO RELATED APPLICATION

This application claims a benefit of priority under 35 U.S.C. 119(e) from co-pending provisional patent application U.S. Ser. No. 63/117,896, filed Nov. 24, 2020, the entire contents of which are hereby expressly incorporated herein by reference for all purposes.

### BACKGROUND OF THE INVENTION

### 1. Field of the Invention

The invention generally relates to near infrared triggered upconversion nano systems for cancer therapy. More particularly, illustrative embodiments are directed to single near infrared irradiation triggered upconversion nano systems for synergistic photodynamic and photothermal cancer therapy.

### 2. Description of the Related Art

As a clinically approved treatment modality, photodynamic therapy (PDT) using a photosensitizer (PS) that can absorb light and convert tissue oxygen into reactive oxygen species (ROS) such as ¹O₂ to kill tumor cells, is an emerging therapeutic modality for cancer treatment. Fullerene has been reported as an efficient photodynamic reagent since 1996. Generally, fullerene could get excited from the ground state to ¹C₆₀ by photoirradiation. This short-lived species is readily converted to long-lived ³C₆₀ via an intersystem crossing. In presence of molecular oxygen, the fullerene can decay from its triplet to ground state, transferring its energy to O₂, generating singlet oxygen ¹O₂.⁵ The absorption of visible or UV light combined with an efficient intersystem crossing to a long-lived triplet state, which makes Fullerene generate reactive oxygen species (ROS), such as hydrogen peroxide, hydroxyl radicals, and superoxides upon illumination, allowing Fullerene to be a photosensitizer for PDT. Compared with original tetrapyrroles PDT groups, (1) Fullerene has a higher ability to produce ¹O₂ (by both type 1 and 2 pathway), (2) Larger Vis-absorbance range in wavelength and cross-section area, (3) Not restricted by tumor hypoxia, (4) More photostable and are less photobleached. However, similar to other conventional photosensitizers applied in PDT, Fullerenes are mostly triggered by short-wavelength light (UV/Visible) and suffered from a limitation of low bio-tissues penetration. The utilization of these traditional photosensitizers is confined to treating topical lesions on the lining of internal organs or cavities or just under the skin and is less efficacious when treating deep-seated and large-size tumors.

### SUMMARY

According to an embodiment of this disclosure, a composition of matter comprises: a nanocomposite comprising a photon upconversion nanoparticle coupled to a photosensitizer nanoparticle that can absorb light and convert tissue oxygen into reactive oxygen species.

According to another embodiment of this disclosure, a low temperature hydrothermal method of making photon upconversion nanoparticles comprises: dispersing Yb(NO₃)₃, Y(NO₃)₃, and Er(NO₃)₃ in water to prepare a mixture; then adding to the mixture an ethylenediaminetetraacetic acid and NaF with sonication to make a solution; then adjusting a pH of the solution to approximately 3.5 using HNO₃ and NaOH; then treating the solution hydrothermally at approximately 130° C. for approximately 4 hours; then quenching to approximately 20° C.; and then collecting and washing the photon upconversion nanoparticles.

According to another embodiment of this disclosure, a near infrared triggered photon upconversion method for synergistic photodynamic and photothermal cancer therapy of a mammal in need thereof comprises: administering to the mammal a nanocomposite comprising a photon upconversion nanoparticle coupled to a photosensitizer nanoparticle that can absorb light and convert tissue oxygen into reactive oxygen species and a photothermal nanomaterial that converts light to heat; and exposing the mammal and the nanocomposite to a near infrared source of actinic radiation.

The descriptions of the various embodiments of the present invention have been presented for purposes of illustration, but are not intended to be exhaustive or limited to the embodiments disclosed. Not all embodiments will include all of the features described in the illustrative examples. Further, different illustrative embodiments may provide different features as compared to other illustrative embodiments. Many modifications and variations will be apparent to those of ordinary skill in the art without departing from the scope and spirit of the described embodiment. The terminology used herein was chosen to best explain the principles of the embodiment, the practical application or technical improvement over technologies found in the marketplace, or to enable others of ordinary skill in the art to understand the embodiments disclosed here.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIGS. 1A-1D are illustrations of (A) SEM image of UCNPs with size average 120 nm; (B) EDS certification of UCNPs; (C) XRD pattern of UCNPs; and (D) Upconverting luminescence with 980 nm radiation 2 W;

FIGS. 2A-2F are illustrations of (A) TEM image of UCNP@silica with size average 140 nm; (B) FT-IR spectrum of UCNPs, UCNP@silica, and UCNP@C60; (C) SEM image of bare AuNRs; (D) UV-Vis absorbance after purification; (E) SEM image of UCNP@C60+AuNRs; and (F) TEM image of UCNP@C60+AuNRs;

FIGS. 3A-3B are illustrations of (A) mechanism of ROS detection of UCNP@C60 through oxidization of DCFH₂; (B) absorbance and fluorescence spectrum of DCF after NIR treatment to UCNP@C60;

FIGS. 4A-4B are illustrations of (A) MCF-7 cell viabilities regarding different radiation time and laser power; (B) Cytotoxicity evaluation from cell viabilities of MCF-7 & MDA-MB-231, with various concentrations of UCNP@silica;

FIGS. 5A-5D are illustrations of in vitro MCF-7 and MDA-MB-231 cell viabilities after incubation with, UCNP@C60 ((A) for MCF-7 and (B) for MDA-MB-231) and AuNRs@PEG@C60 ((C) for MCF-7 and (D) for MDA-MB-231) at different concentrations with/without 980 nm radiation;

FIGS. 6A-6D are illustrations of in vitro MCF-7 and MDA-MB-231 cell viabilities after incubation with, UCNP@C60+AuNRs ((A) for MCF-7 and (B) for MDA-MB-231); Cell viability comparison with cell viability between PDT (AuNRs@PEG), PDT (UCNPs@C60), and PDT+PTT (UCNPs@C60+AuNRs) ((C) for MCF-7 and (D) for MDA-MB-231);

FIGS. 7A-7E are illustrations of (A) changes in the body weights; (B) the relative tumor volume achieved from mice with varying treatments; (C) photographs of excised tumors from representative mice; (D) H&E stained images of tumor tissues obtained after 14 days of treatment; and (E) thermal imaging with/without NIR radiation with varying treatments;

FIG. 8 is an illustration of a principle of the synergistic photothermal and photodynamic of the upconversion therapeutic system;

FIG. 9 is an illustration of a reaction route of nanocrystal NaYF4:Yb/Er synthesis, UC NPs, and AuNRs surface functionalization, and final one-pot conjugation reaction of UCNPs @C60+AuNRs;

FIG. 10 is an illustration of a figure S1 showing XRD analysis of the UCNPs compared with standard data of Cubic phase NaYF4,Yb,Er (JCPDS No. 77-2042);

FIG. 11 is an illustration of a scheme S1 showing synthesis pathway of Carboxyl C60;

FIG. 12 is an illustration of a figure S2 showing ¹H-NMR characterization of the synthesized Carboxyl C60;

FIG. 13 is an illustration of a figure S3 showing MALDI-TOF characterization for synthesized Carboxyl C60;

FIG. 14 is an illustration of a flow chart showing a low temperature hydrothermal methodology for making photon upconversion nanoparticles; and

FIG. 15 is an illustration of a flow chart showing a near infrared triggered photon upconversion methodology for synergistic photodynamic and photothermal cancer therapy of a mammal.

### DETAILED DESCRIPTION

The application of NIR laser to PDT can achieve deeper penetration than that of UV/visible light because most biomolecules absorb minimally in the NIR range; thus, topically transducing NIR laser to visible light is extremely desired for this technology.

Fortunately, tremendous progress in rare-earth-based upconversion nanoparticles (UCNPs) provides an alternative way for the approach of NIR-to-visible conversion. Photon upconversion (UC) is an anti-Stokes type emission process in which the sequential absorption of two or more photons leads to the emission of light at a shorter wavelength than the excitation wavelength. Lanthanide-doped upconversion nanoparticles that can convert low-energy near-infrared light to high-energy ultraviolet (UV)/visible light have received much attention in a variety of biomedical fields. Owing to outstanding luminescence properties, UCNPs have found critical utilities in biomedical fields, including multimodal imaging, controlled drug release, and topical PDT. Recently, a few studies have been published to combine UCNPs with Fullerene as a sound PDT strategy, since 2013. However, its highest efficiency ratio is hitherto about 5% of the whole laser energy, because of its cross-sectional area, and transmission efficiency in the UCNPs itself. Considering these, we designed a combination of PDT and PTT, with an enhanced laser therapeutic efficiency with more than 52% IC₅₀ decrease. Compare with traditional therapy, combination therapy, based on simultaneously using two or more types of therapies, provides a potential solution for cancer treatment because of its synergistic enhancement. In recent studies, multimodal therapies becomes a hot-spot, and a promising strategy of combining PDT and PTT showed an excellent potential to ablate tumors. Gold Nanorods (AuNRs) are identified to be a best for PTT, because its almost 98% absorption efficiency and easy modulated localized surface plasmon resonance (LSPR) peaks, compared with other nanodevices. Therefore, we reason that, UCNPs together with Fullerene and AuNRs, as a PDT and PTT modality may provide a powerful toolbox for synergistic phototherapy.

The development of single near-infrared (NIR) laser-triggered phototherapy and multimodal combined therapy is highly desirable but is still a big challenge. Although Fullerenes have shown great potential as photodynamic therapy (PDT), the use of such Fullerene in PDT is limited by the shallow depth of tissue penetration of short-wavelength light. Therefore, to combat such limitations, we rationally designed a NIR triggered upconversion nanoparticle@C60 (UCNP-C60) with Gold Nanorods (AuNRs) for combinational synergistic phototherapy, results in better treatment outcomes other than monomodal photodynamic therapy (PDT) or photothermal therapy (PTT). Herein, NaYF₄:Yb/Er Upconversion Nanoparticles (UCNPs) were rationally synthesized via a novel low-temperature hydrothermal method, exhibiting excellent photoluminescence emissions, under NIR laser radiation. After surface modification with silanization and amine terminals decoration, UCNPs were carbodiimide coupling grafted with benzoyloxy pyrrolidine grafted C60 derivatives, as photosensitizers. Meanwhile, carboxylic polyethylene glycol (PEG) functionalized AuNRs with localized surface plasmon resonance (LSPR) at 980 nm were covalently conjugated with the UCNP-C₆₀ nanocomposite to obtain a multifunctional nanoplatform for a synergistic PTT and PDT. Notably, under NIR laser irradiation, ¹O₂ was effectively generated from an upconverting photodynamic combination of UCNPs and C60, while localized hyperthermia was simultaneously induced by LSPR activity of AuNRs. Its therapeutic efficacy was demonstrated in vitro on breast cancer cell MCF-7 and MDA-MB-231, and in vivo on 4T1 cell inoculated mice, under a significantly mild NIR irradiation and low dosage of the nanocomposite. Furthermore, according to cell viability comparative analysis, UCNP-C₆₀-AuNRs presents remarkable synergistic therapeutic effects by integration of PTT and PDT, with 53% viabilities decrease. This work highlights an innovative strategy for the design and understanding of clinical phototherapeutic, which has the potential of conquering the extreme heterogeneity and complexity in oncotherapy.

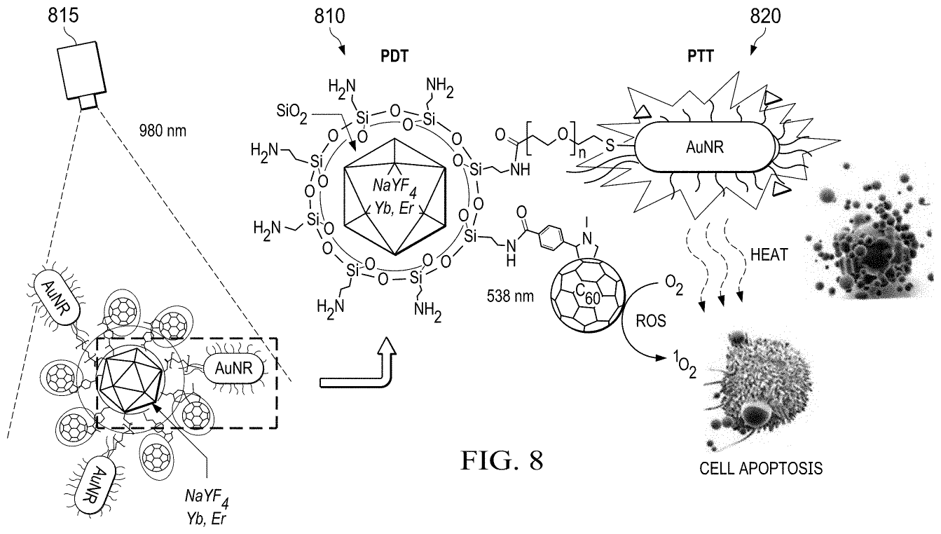

FIG. 8 shows a synergistic photothermal 820 and photodynamic 810 upconversion therapeutic system. A near infrared laser 815 provides actinic radiation at approximately 980 nm. This is upconverted to approximately 538 nm and converts tissue oxygen into reactive oxygen species. Meanwhile the gold nanorods provide photothermal therapy.

To validate our hypothesis, a novel upconverting system was synthesized with NaYF₄:Yb/Er nanocrystals was rationally synthesized through a low-temperature hydrothermal method, exhibiting strong yellowish-green photoluminescence emission under 980 nm laser radiation. The UCNPs were then surface modified with silanization of tetraethyl orthosilicate (TEOS) and aminopropyltrimethoxysilane (APTMS) for water solubility improvement and amine terminals decoration. Subsequently, via a carbodiimide coupling reaction, UCNPs were grafted with a photosensitizer—a benzoyloxy pyrrolidine based C60 derivative, which was functionalized through the Prato reaction. Thereafter, the carboxylic polyethylene glycol (PEG) functionalized AuNRs (LSPR peak 980 nm), along with photosensitizer C60, were covalently conjugated around the UCNPs to obtain a multifunctional strategy for simultaneous PTT and PDT. Notably, under single NIR laser irradiation, ¹O₂ was effectively generated from UCNPs-C60 as an upconverting photodynamic combination, while localized hyperthermia was simultaneously induced by LSPR activity of AuNRs, as shown in FIG. 8. Its therapeutic efficacy was validated in vitro on breast cancer cell lines MCF-7 and MDA-MB-231, and in vivo on 4T1 cell inoculated mice, by various microscopic and biochemical studies under a significantly mild NIR irradiation and low dosage of the nanoplatforms. Furthermore, according to cell viability and tumor volume comparative analysis, such nanocomposite presents a remarkable synergistic therapeutic effect by the combination of PTT and PDT, with a more than 51% IC₅₀ decrease. Overall, this work provides an innovative strategy for the design and understanding of clinical phototherapeutic and photo-controlled drug release.

Experimental Section

Materials

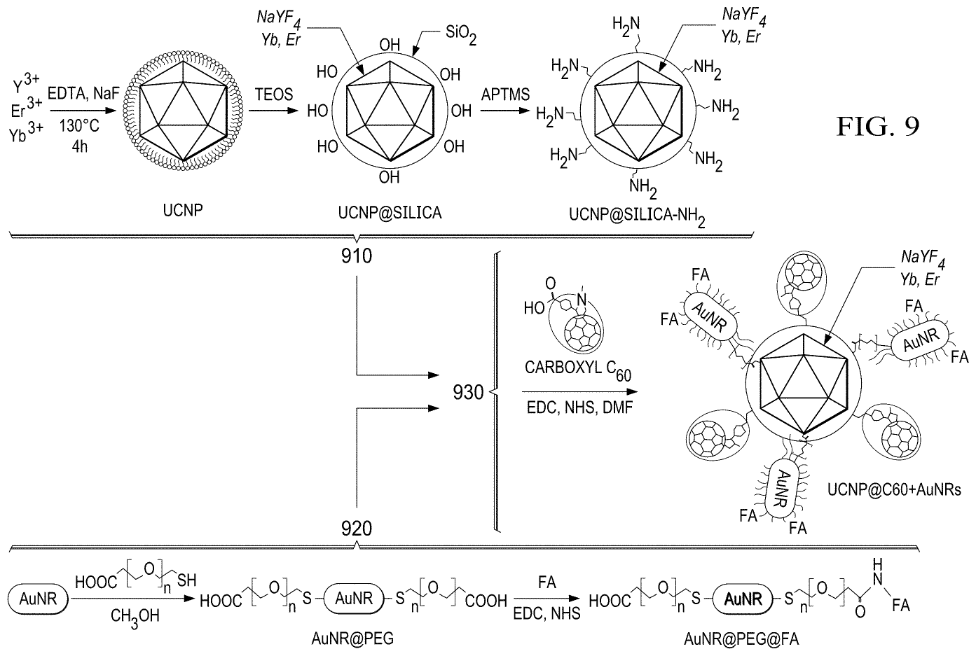

FIG. 9 shows a reaction route 910 of nanocrystal NaYF4:Yb/Er synthesis of UCNPs, AuNRs surface functionalization 920, and final one-pot conjugation reaction of UCNPs @C₆₀+AuNRs 930.

Sodium fluoride (NaF), ammonium fluoride (NH₄F), Yb(NO₃)₃ (99.8%), Y(NO₃)₃ (99.5%), Er(NO₃)₃ (99.5%), hydrochloric acid (HCl), ethylenediaminetetraacetic acid (EDTA), cyclohexane, N,N-dimethylformamide (DMF), sodium hydroxide (NaOH), tetraethyl orthosilicate (TEOS), chloroauric acid (HACl₄), sodium citrate (HOC(COONa)(CH₂COONa)₂), sodium borohydride (NaBH₄), ascorbic acid, and hexadecyltrimethylammonium bromide (CTAB), (from Thermal Fisher Co., Ltd.), oleic acid (OA), 1-octadecene (ODE), N-hydroxysulfosuccinimide (Sulfo-NHS), 1-(3-(dimethylamino)propyl)-3-ethylcarbodiimide hydrochloride (EDC.HCl), aminopropyltrimethoxysilane (APTMS), folic acid (FA), 2′,7′-dichlorofluorescein diacetate (DCF-DA) and calcein AM (from Sigma-Aldrich. Co. LLC); Fullerene C60 (Carbon 60) (SES Research Inc., Houston, Tex.), and thiol PEGylated carboxylic acid (HS-PEG-COOH) (from Sigma Co., Ltd.) were analytical grade without purification. All aqueous solutions were prepared using ultrapure water purified by a Milli-Q system (Millipore, Bedford, Mass., U.S.A.).

Synthesis and Characterization

Synthesis of UCNPs: NaYF4:Yb/Er Nanocrystal and Surface Modification.

The UCNPs were synthesized as follows: 0.846 mmol of Yb(NO₃)₃, 3.3 mmol of Y(NO₃)₃, and 0.089 mmol of Er(NO₃)₃ were dispersed in 40 mL Milli-Q water with ultrasonic and stirring. Then 3.3 mmol of EDTA (ethylenediaminetetraacetic acid) and 20 mmol of NaF were added and the mixture was sonicated for 20 min, transferred to a 50 mL Teflon-lined stainless-steel autoclave. After adjusting the pH of the solution to 3.5 with HNO₃ and NaOH, the autoclave was sealed, and then hydrothermally treated at 130° C. for 4 h. After the autoclave was quickly cooled down to room temperature with a water rinse, the UCNPs were collected and washed with deionized water and then ethanol three times.

For the surface coating process, briefly, 20 mg synthesized UCNPs were dispersed into 10 mL ethanol and ultrasonicated for 10 min. Afterward, ammonium hydroxide (28 wt %, 1 mL) and TEOS (40 uL) were subsequently added drop wisely during stirring at 800 rpm using a mini-stir bar at room temperature. Then, the solution was treated with ultrasonication for 3 h, with stirring. The final sample was centrifuged at 13,000 rpm for 10 min to obtain UCNPs embedded silica nanoparticles. After washing with deionized water and ethanol twice, the nanoparticles were redispersed in 3 ml of ethanol, and APTMS (20 μL) was added dropwise. The mixture was stirred at 1200 rpm using a mini-stir bar for 12 h at room temperature to obtain the APTMS-coated UCNPs (UCNP @ Silica).

Synthesis of Carboxyl Functionalized Fullerene Derivative

The carboxyl functionalized Fullerene derivative was synthesized through a typical Prato reaction (Scheme S1). Briefly, a mixture of 4-carboxybenzaldehyde (0.210 g, 1.40 mmol), C₆₀ (0.202 g, 0.28 mmol), and N-methylglycine (0.125 g, 1.40 mmol) in chlorobenzene (60 mL) was refluxed overnight under a nitrogen atmosphere. The solvent was removed by rotary evaporation under reduced pressure. The crude product was purified over silica gel column chromatography with toluene to toluene/THF (2/1) as the eluents to afford a brown-yellow solid (0.098 g, 37%). ¹H NMR (300 MHz, DMSO-d₆): δ 2.20 (s, 3H), 6.65 (s, 1H), 6.89 (s, 2H), 8.03 (d, J) 8.4 Hz, 2H), 8.15 (d, J) 8.4 Hz, 2H), 10.12 (s, 1H). Calculated for C₇₀H₁₁NO₂: C, 93.64; H, 1.23; N, 1.56. Found: C, 93.45; H, 1.31; N, 1.62. ESI-MS (m/z): calculated, 897.1; found, 897.0.

Synthesis of AuNRs, Purification, and PEG Modification.

At room temperature (ca. 25° C.), 100 μL HACl₄ (50 mM) was added to 20.0 mL of sodium citrate (0.25 mM). 600 μL of a freshly prepared NaBH₄ (100 mM) solution was rapidly injected under vigorous stirring (>1400 rpm). After 2 min, to prepare the Au seed solution, the mixture was kept under mild stirring (400 rpm) for 40 min at room temperature and 15 min at 40-45° C. before use (orange-red color). Then 12.5 μL of 0.05 M HAuCl₄ was added to a mixture of 3 mL of water and 2 mL of 0.1M CTAB. The solution was cooled down to 22° C. in a thermostatic bath. 12.5 μL of 0.1 M ascorbic acid was then added to the solution and shaken by hand; the mixture turned colorless in a few seconds. Finally, 835 μL of the Au seed in a citrate solution was added, shaken by hand, and left undisturbed for 3 hours at 22° C. Then CTAB (4 mL 0.1M) was added to 40 mL of water. 60 μL of HAuCl₄ 0.05 M solution was then added, the solution was gently shaken and cooled down to 20° C. in a thermostatic bath. Subsequently, 75 μL of 0.1 M ascorbic acid solution was added to the mixture, and the solution was gently shaken until it turned completely colorless. Finally, 65 μL seeds solution was added to the growing mixture; the solution was vigorously shaken by hand and then left undisturbed overnight at 20° C. For the process of purification, the raw AuNRs colloidal solution from the growth step was taken centrifugation at 6000 rpm for 5 min. The pink precipitate containing rods and large spheres was collected from each centrifuge tube and the pink supernatant containing small spheres and surfactant was discarded. All the precipitates collected from 50 mL solution were dissolved in 10 mL of 0.1M hot (40-50° C.) CTAB solution. Brown precipitate along with pink supernatant was observed upon cooling at room temperature. The precipitate was separated from the supernatant and again dissolved in a fresh 10 mL of 0.1 M hot (40-50° C.) CTAB solution. After cooling to room temperature, the precipitate was collected, while the supernatant was added to the previous supernatant. This precipitation and redispersion can be repeated many times for reaching high purity. In the present case, we generally repeated 3 times for the complete separation of long rods. The precipitate was finally dissolved in 10 mL distilled water and stored for further reactions. Characterizations were taken by SEM and UV-Vis spectrum.

After purification, a PEG surface functionalization was introduced. The AuNRs were centrifuged, washed with water 2 times to de-coat CTAB, and dispersed with 10 ml ethanol. Then HS-PEG-COOH was added dropwise with stirring. The products were collected with centrifuge after reacting overnight. Afterward, folic Acid was partially conjugated with a simple EDC-NHS carboxyl activator reaction. The obtained acid-terminated surface was activated by a reaction with NHS in the presence of a peptide-coupling agent EDC and then reacted with the amino linker of the Folic acid to anchor the surface at PEG-coated AuNRs by a covalent amide bond. 5 mg AuNRs were dissolved in 10 mL methanol and added into a 20 mL vial with stirring at room temperature. Then 10 mg EDC was added to the mixture with stirring for 15 min. After fully dissolved in methanol, 4 mg NHS-sulfo was added to the mixture with stirring for another 40 min, for the complete replacement of EDC. Finally, 2 mg FA was added to the mixture and stirred at room temperature overnight. The product named AuNRs@ PEG @ FA was further purified with methanol washing and centrifugation 3 times.

Carboxyl Active Conjugation with UCNP@Silica, Carboxyl C₆₀, and AuNRs@PEG@FA

The conjugation with UCNP@Silica, Carboxyl C₆₀, and AuNRs@PEG@FA, was processed with a one-pot EDC-NHS reaction, with a similar procedure as carboxyl activator reaction. The obtained acid-terminated surface of Carboxyl C₆₀ and AuNRs@PEG@FA were activated by a reaction with NHS in the presence of a peptide-coupling agent EDC and then reacted with the amino linker from the surface of UCNP @ Silica, for anchoring the surface by a covalent amide bond. 6 mg of AuNRs@PEG @FA and 1 mg of Carboxyl C₆₀ were dissolved in 10 mL DMF and added into a 20 mL vial with stirring at room temperature. Then 10 mg EDC was added to the mixture with stirring for 15 min. After fully dissolved in DMF, 4 mg NHS-sulfo was added to the mixture with stirring for another 40 min, for the complete replacement of EDC. Finally, 20 mg UCNP@Silica-NH₂, was added into the mixture and stirred at room temperature overnight. The product named UCNPs @C₆₀+AuNRs was further purified with DMF washing and centrifugation for 3 times.

Nanomaterial Characterization

Upconversion emission spectra were measured on an apparatus using an infrared diode 980 nm Laser (MDL-H-980/3000-5000 mW, Opto Engine LLC, Utah, USA) as the excitation source. UV-Vis absorption spectra were acquired by SpectraMax M3 Microplate Readers. Fourier-transform infrared (FT-IR) spectra were tested on a Vertex PerkinElmer 580BIR spectrophotometer (Bruker), using the KBr pellet technique. X-ray diffraction patterns of the particles were collected on a D8 Focus diffractometer (Bruker) using CuKα radiation (λ=0.15405 nm). The samples for XRD analysis were prepared by depositing the nanoparticle solution on the glass slides and drying at 80° C. in a vacuum. Scanning electron microscopy (SEM) and energy-dispersive X-ray (EDS) were obtained from a Hitachi S4800 field emission. Transmission electron microscopy (TEM) micrographs were obtained from an FEI Tecnai G2 S-twin transmission electron microscope with a field emission gun operating at 200 kV. Cell viabilities images were obtained through Nikon Eclipse Ti Fluorescence Microscope. The upconverting luminescence was detected with USB4000 UV-NIR Spectrometer from Ocean Optics, Inc.

Upconverting Luminescence from UCNPs and Evaluation of ROS

The upconverting emission of the UCNPs was tested with the OceanOptic spectrum. The excitation was processed in a dark room with 980 nm Laser treatment at 2 W. After excitation, four main peaks were emitted 525 nm, 540 nm, 653 nm, and 839 nm, respectively. After the conjugation of UCNPs with C60, the ROS generation efficiency was tested with a pre-fluorescent and reductive reagent DCFH₂, which has the property of easy to get oxidation with ROS. Typically, 1 mL of sample solution (1 mg/mL) was mixed with 1 mL of ethanol and put into a 2 mL vial. The solution was kept in the dark and irradiated by a 980 nm laser resource (1.5 W/cm²) for 2 min. Then the supernatant was collected for UV-Vis measurements. The intracellular ROS generation ability of the UCNPs@C60 was studied through DCFH-DA. After oxidization, the DCFH-DA was converted into DCF with a big π conjugated structure, which made a huge absorbance difference (into 501 nm) and fluorescence at 521 nm.

Cell Culture

MCF-7 (ATCC Cat No. HTB-22) & MDA-MB-231 (ATCC Cat No. HTB-26) cell line was purchased from the American type culture collection and maintained in DMEM medium containing 10% FBS (Invitrogen, Burlington, Canada). All of the cell lines were cultured under a humidified atmosphere of 5% CO₂ at 37° C.

In-Vitro Cytotoxicity and Anticancer Activity Evaluation

For the cytotoxicity test (cell proliferation assay), MCF-7 & MDA-MB-231 breast cancer cells (6000˜7000/well) were seeded into a 96-well plate and incubated at 37° C. with 5% CO₂ for 24 h to obtain monolayer cells. Samples with various concentrations of UCNP@C₆₀+AuNRs, UCNP@C₆₀, UCNP@Silica, and AuNRs@PEG were tested. The samples were diluted into the wells with various concentrations (UCNP@C₆₀+AuNRs: 0, 4.95, 23.81, 45.45, 83.33, 115.38, 142.86, and 166.67 ng/mL; UCNP@C₆₀: 0, 37.4, 73.7, 143, 216, 275, 320, 423, and 550 ng/mL; UCNP@Silica: 0, 9.90, 19.61, 47.62, 90.91, 166.67, 230.77, 285.71, 384.62, and 566.04 ng/mL; AuNRs@PEG: 0, 0.40, 1.98, 3.92, 6.76, 9.52, 13.95, 18.18, 33.33, 57.14, 66.67, and 86.67 ng/mL) and incubated for another 24 h. Samples with each concentration were introduced without 980 nm laser radiation. Thereafter, 50 μL of dye solution (5 μL EthD-1 and 0.5 μL Calcein AM in 1 mL PBS (pH 7.4)) was added to each well. After incubation for 1 h, the fluorescent data of each well were obtained by Fluorescence Microscope (Nikon Eclipse Ti, Nikon Instruments Inc), and Cell viabilities of each well were detected and analyzed through ImageJ via particles analysis.

For anticancer activity assay, MCF-7 & MDA-MB-231 breast cancer cells (60/007,000/well) were seeded into 96-well plates separately and incubated at 37° C. with 5% CO₂ for 24 h to obtain monolayer cells. Among them, 8 wells were left with culturing only for the control group. The samples were diluted into various concentrations (same as cytotoxicity tests) and incubated for another 24 h. Samples with each concentration were introduced with 980 nm laser radiation for 2 min with 1.5 W/cm². Thereafter, 50 μL of EthD-1 and Calcein AM solution (5 μL EthD-1 and 0.5 μL Calcein AM in 1 mL PBS (pH 7.4)) was added to each well. After incubation for 1 h, the fluorescent data of each well were obtained by Fluorescence Microscope (Nikon Eclipse Ti, Nikon Instruments Inc), and Cell viabilities of each well were detected and analyzed through ImageJ via particles analysis.

In-vivo Phototherapy Evaluation.

The 4T1 cells were injected subcutaneously in the left axilla of each mouse (about 20 g) to obtain tumors. When the tumors grew to 6˜8 mm (60˜100 mm³), the mice were randomly divided into 4 groups (6 mice per group, minimizing the differences of weights and tumor sizes in each group) and injected in vein with the saline, UCNP@C₆₀+AuNRs, and UCNP@C₆₀, respectively. And as-synthesized samples (20 mg/kg in 0.2 mL PBS) were injected into tumor-bearing mice three times on days 1, 3, and 5, respectively. The tumor focus was irradiated with a 980 nm laser (every time after nanomedicine administration) for 1.5 W cm⁻² with 10 min (2 times of 5 min irradiation with 5 min intervals). The mice were observed daily for clinical symptoms and the tumor sizes were measured by a caliper every other day and calculated as the volume=(tumor length)×(tumor width)²/2. After treatment for 17 days, the mice were sacrificed to collect tumors for H&E staining. Morphological changes were observed under the microscope.

The histological analysis was carried out after 2 weeks of treatment. Less than 1 cm×1 cm of tissues of each representative tumor tissue of the mice in 4 groups were excised. Then the excised tissues were successively dehydrated using buffered formalin, ethanol of various concentrations, and xylene. Thereafter, the dehydrated tissues were embedded in liquid paraffin and sliced to 3×5 mm for hematoxylin and eosin (H&E) staining. The final stained slides were observed using an optical microscope (Nikon TS100).

Results and Discussion

Preparation and Characterization of UCNPs, UCNP@C60, and AuNRs

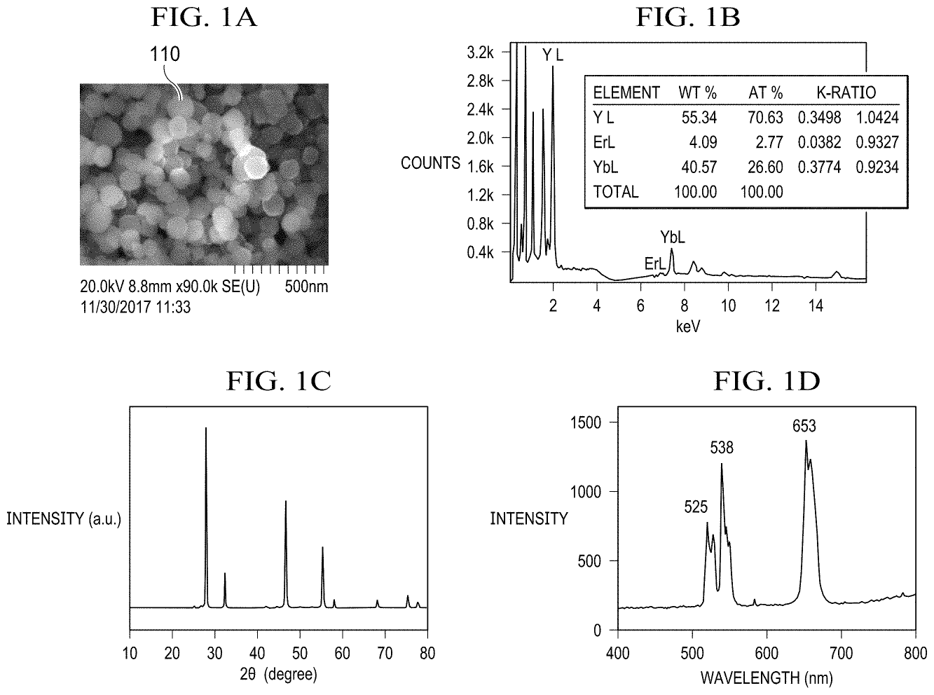

FIG. 1A shows an SEM image of UCNPs 110 with a size average of approximately 120 nm. FIG. 1B shows EDS certification of UCNPs. FIG. 1C shows an XRD pattern of UCNPs. FIG. 1D shows upconverting luminescence with 980 nm radiation 2 W.

The UCNPs (NaYF4:Yb,Er) were prepared by a novel hydrothermal decomposition method. Compared with the original oleic acid-mediated method, this hydrothermal method used EDTA as the surfactant, which has a much better water solubility than oleic acid, which means cutting down multiple steps of surfactant exchange and washing process, before surface coating and modification process for biomedical utilization. It is worth mentioning that this hydrothermal method (130° C.) provides a new way to fabricate 100 nm level UCNPs through a much lower reaction temperature than the normal oleic acid method, which is ˜300° C. In the SEM image of UCNPs (FIG. 1A), the sample consists of uniform and monodisperse nanoparticles with an average diameter of ˜120 nm. The SEM image (FIG. 1A) indicates that the uniformity and dispersity of the UCNPs have been kept well. EDS spectrum (FIG. 1B) verify the elemental composition of UCNPs, with two peaks of 6.974 KeV excited from La of Er and 7.414 KeV excited from La of Yb. And the corresponding occupancy of Er and Yb from the NaYF₄ are 2.77% and 26.60% respectively. The crystallographic form of UCNPs was tested through XRD, as shown in FIG. 1C. The XRD pattern showed an excellent match with cubic phase α-NaYF₄, Yb, Er crystal, with the comparison of JCPDS No. 77-2042 (Figure S1). Thereafter, the upconverting emission of the UCNPs was tested with the UV-NIR Spectrometer (FIG. 1D). The excitation was processed in a dark room with 980 nm Laser treatment at 2 W. After excitation, four main peaks were emitted 525 nm, 540 nm, 653 nm, and 839 nm, respectively. According to the result, these characteristic emission peaks of Er³⁺ correspond to ²H_{11/2}—⁴I_{15/2} (510-530 nm), ⁴S_{3/2}-⁴I_{15/2} (530-570 nm), and ⁴F_{9/2}-⁴I_{15/2} (630-680 nm) transitions were verified. These results confirm the successful upconverting luminescence from the synthesized UCNPs.

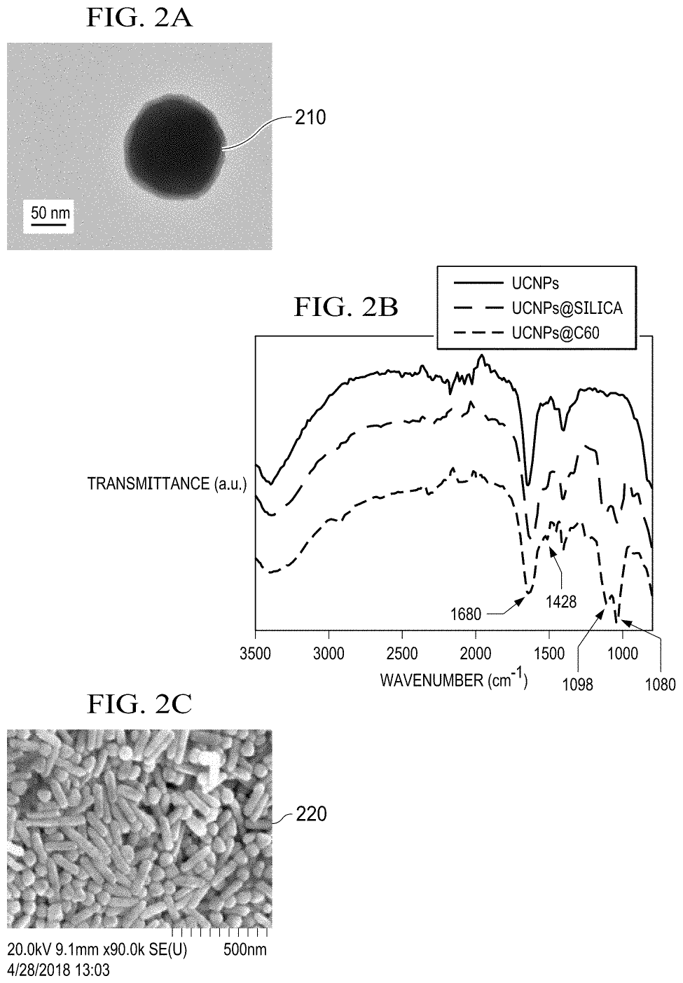

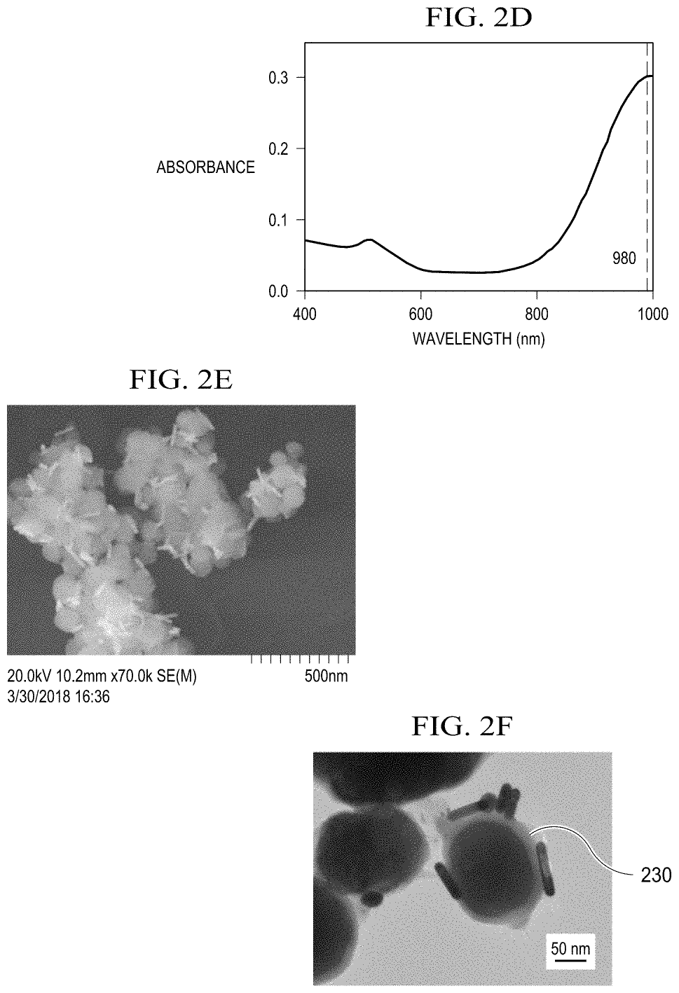

FIG. 2A shows TEM image of UCNP@silica with a size average 210 of approximately 140 nm. FIG. 2B shows an FT-IR spectrum of UCNPs, UCNP@silica, and UCNP@C60. FIG. 2C shows an SEM image of bare AuNRs 220. FIG. 2D shows UV-Vis absorbance after purification. FIG. 2E shows an SEM image of UCNP@C60+AuNRs. FIG. 2F shows a TEM image of UCNP@C60+AuNRs 230.

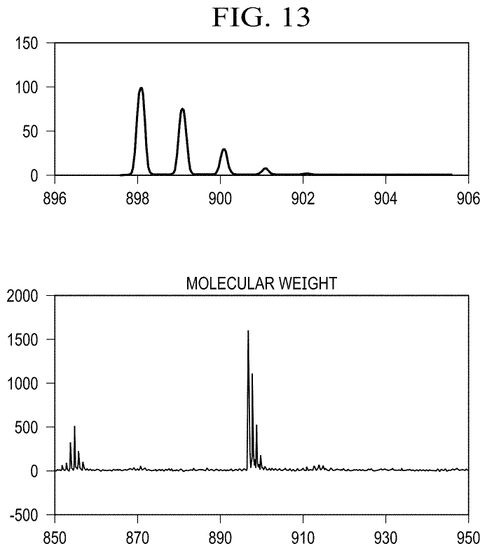

After the UCNPs were collected and washed through centrifugation and sonication, UCNPs were first introduced to TEOS for surface silanization. Therefore, the UCNPs solubility was greatly increased. Then UCNPs were introduced with further modification of APTMS, from which amino terminals were grafted, for preparation of further conjugation purposes. The product was named UCNPs @ silica. Notably, from FIG. 2A the uniformity and dispersity are retained very well after silanization treatment, and its size is slightly increased compared with bare UCNPs. As expected, compared with bare UCNPs, a clear semitransparent 10 nm shell can be obtained from TEM imaging, as shown in FIG. 2A. The photosensitizer C60 was carboxyl functionalized through a modified noble Prato reaction, using commercial Fullerene. The product Carboxyl C60 was characterized with NMR and Mass spectrum (FIGS. 12-13), with a yield of 37%. Then covalently amido conjugated on the surface of UCNPs, through a classic EDC-NHS catalyzed carboxyl activation reaction. The UCNPs with silica shell and C₆₀ was named UCNP@C60. FT-IR spectroscopy was used to identify the functional groups on the surface and give additional evidence for successful modification, and the FT-IR spectra of UCNPs, UCNP@silica, and UCNP@C₆₀ are presented in FIG. 2B. As shown, the strong transmission bands at 1098 and 1080 cm⁻¹ are assigned to the asymmetric and symmetric stretching vibrations of silica (Si—O), respectively, and another band at around 1680 cm⁻¹ originates from the C═O stretching vibration from amino linkage and DMF solvent. After C₆₀ conjugation, a stronger transmission band at 1428 cm⁻¹ can be found which assigns dipole active vibrational of C₆₀. These results confirm the successful silica coating and C60 conjugation.

The other important component AuNRs was synthesized through a classical seed-mediated method. Its morphology and NIR absorbance were characterized through SEM and UV-Vis, as shown in FIG. 2 C&D. In the SEM image of AuNRs (FIG. 2C), the sample consists of uniform and monodisperse nanorods with an average diameter of ˜25×100 nm. The SEM image indicates that the uniformity and dispersity of the UCNPs have been kept well. And from the UV-Vis-NIR absorbance (FIG. 2D), two obvious peaks 520 nm and 980 nm, can be obtained, which belong to peaks of SPR and LSPR, respectively. It is worth mentioning that, the high LSPR peak and the high absorbance ratio of Abs_{980 nm}/Abs_{525 nm} indicated that a high photothermal efficiency.^{17, 21} Thereafter, the AuNRs were introduced with template replacement and conjugation with thiol PEG through a noble Au—S bond. And the carboxyl terminals were partially conjugated with a trace amount of targeting group FA via an EDC-NHS conjugation. The final product UCNP@C₆₀+AuNRs was synthesized through a similar EDC-NHS conjugation as well, and the product was purified with 3 times of methanol washing and redispersion. The morphology of the final product UCNP@C₆₀+AuNRs was detected with SEM and TEM, as shown in FIG. 2 E&F. The SEM image (FIG. 2 E) indicates that the uniformity and dispersity of the UCNP@C₆₀+AuNRs have been kept well. The AuNRs@PEG were well-proportioned conjugated on the surface of UCNP@C₆₀. However, from SEM we can not find a single UCNP@C₆₀+AuNRs, therefore TEM was introduced for imaging the single UCNP@C₆₀+AuNRs, as shown in FIG. 2 F. In the TEM image, I clear monodispersed UCNP@C₆₀+AuNRs was obtained, with multiple AuNRs conjugated on a single UCNPs. It is worth mentioning that, in FIG. 2F, a few spherical at an average size of 25 nm can be obtained surrounding the UCNP core. Regarding their size, we assume they should be AuNRs at directions of perpendicular to the field of view, therefore only the intersecting surface can be obtained from the AuNRs. Another assumption is they might be gold nanoparticles (AuNPs), from the imperfect AuNRs purification, where still a small portion of AuNPs may exist.

ROS Generation from NIR Irradiation with UCNP@C60

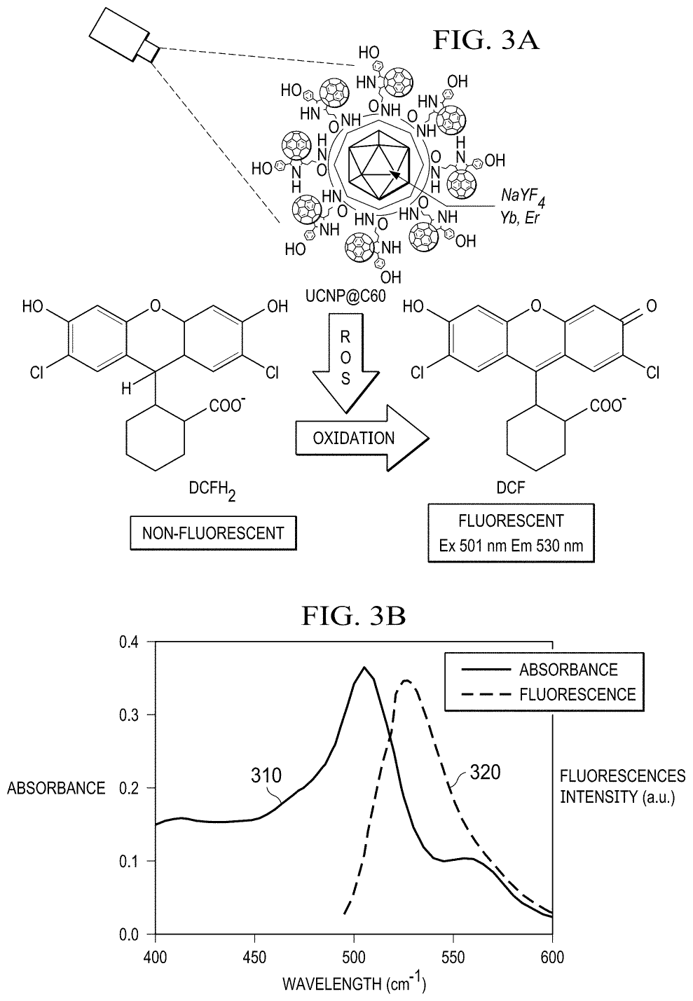

FIG. 3A shows a mechanism of ROS detection of UCNP@C60 through oxidization of DCFH₂. FIG. 3B shows absorbance spectrum 310 and fluorescence spectrum 320 of DCF after NIR treatment via UCNP@C60.

After the radiation of NIR, the ROS generation efficiency of UCNP@C60 was verified with a pre-fluorescent and reductive reagent DCFH₂, which has a property of easy oxidation with ROS (FIG. 3A). The DCFH-DA can be taken up by cells but cannot radiate fluorescence. However, the DCFH molecules could be oxidized to DCF with luminous green fluorescence under 488 nm light radiation after hydrolysis to DCFH by intracellular esterase. FIG. 3B exhibits the UV-Vis and fluorescent spectra of UCNP@C60 with DCFH-DA. After 2 min 1.5 W/cm² of 980 nm laser radiation, a strong green fluorescence also indicates that the significant ROS generation of the UCNP@C60 upon 980 nm laser irradiation. As shown in FIG. 3B, the oxidized DCF has a big π conjugated structure, which made a huge absorbance difference (into 501 nm) and fluorescence at 521 nm.

Laser Parameter Optimization

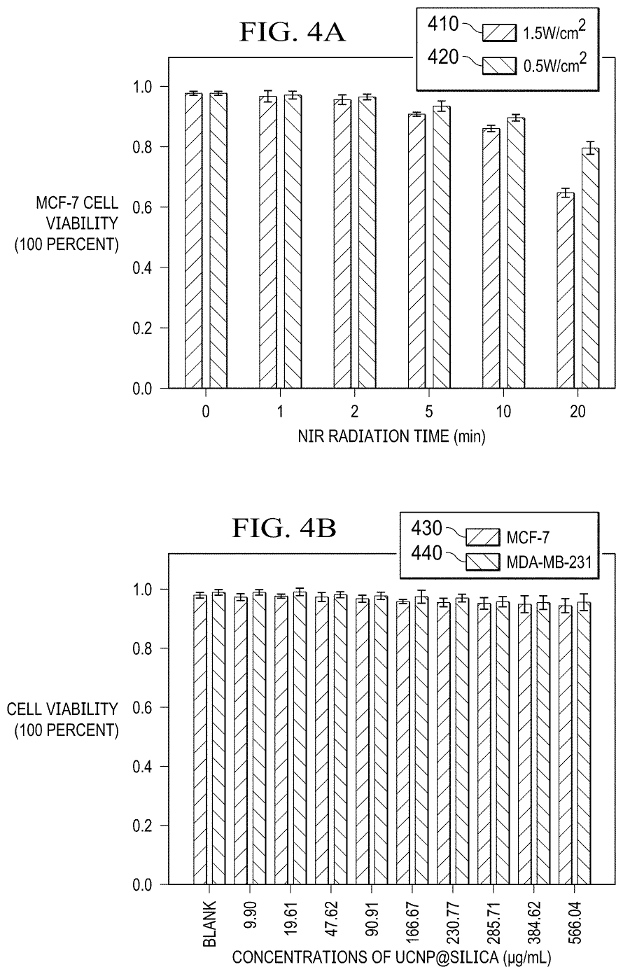

FIG. 4A shows MCF-7 cell viabilities regarding different radiation times and laser powers 1.5 W/cm² 410 (left) & 0.5 W/cm² 420 (right). FIG. 4B shows cytotoxicity evaluation from cell viabilities of MCF-7 430 (left) & MDA-MB-231 440 (right), with various concentrations of UCNP@silica.

Before in vitro utilization, it is necessary to assess the optimization of the laser radiation parameters. Regarding this, two main parameters were introduced and optimized, which are laser power and radiation time. During the optimization, with 980 nm laser radiation, the cell viabilities of MCF-7 were tested, regarding varieties of laser power and radiation duration. As shown in FIG. 4 A, when laser power is at 0.5 or 1.5 W/cm², which has been well explored in many studies, showed up no significant cell viability decrease before 2 min. However, after 2 mins, samples with 1.5 W/cm² can be detected about 5% viability decrease, while 3% for samples with 0.5 W/cm². To minimize the unexpected heating increase from 980 nm laser radiation (as water has an absorbance at 980 nm wavelength), 2 min radiation time was chosen, and used for the following experiments. However, since a higher laser power indicates a bigger photon currency, indicating a better therapeutic efficiency, a higher laser power was expected. Therefore, regarding comprehensive consideration of the radiation time and tissue damage, from this graph, 2 min, 1.5 W/cm² was chosen for the following assays.

Cell Viabilities for Cytotoxicity of UCNPs

Before practical utilization, it is necessary to assess the biocompatibility of the as-fabricated products. FIG. 4B showed the MCF-7 and MDA-MB-231 cell viabilities after incubating with the UCNP@silica samples in various concentrations were tested by EthD-1&Calcein assays. As shown in FIG. 4B, both MCF-7 and MDA-MB-231 cells showed viabilities of higher 94.1˜95.2% in the entire concentration range even with UCNP@silica >500 μg/mL, indicating the excellent biocompatibility of UCNP@silica.

PDT & PTT Evaluations for UCNP@C60 and AuNR@PEG with/without NIR Radiation

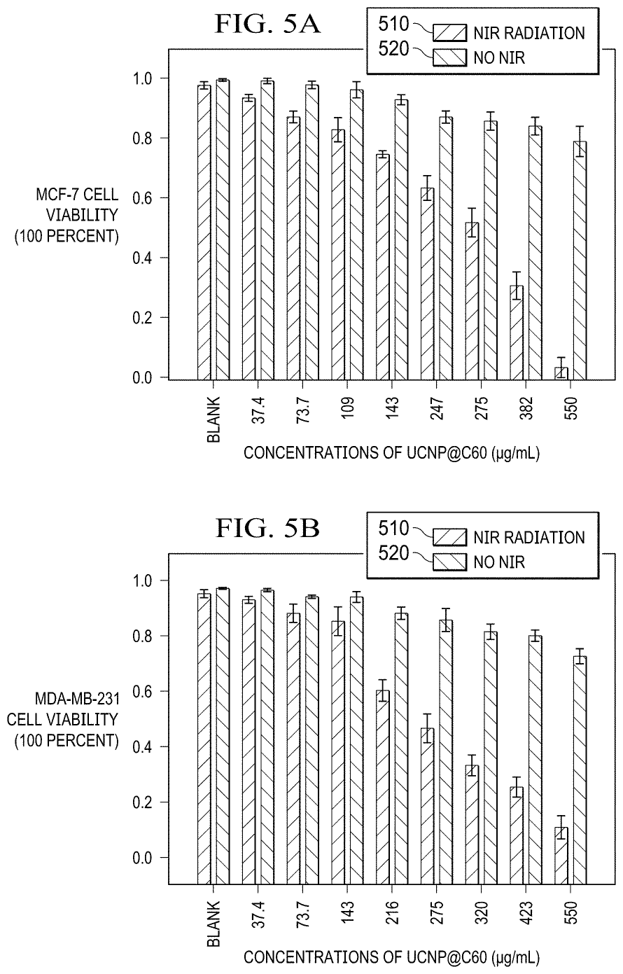

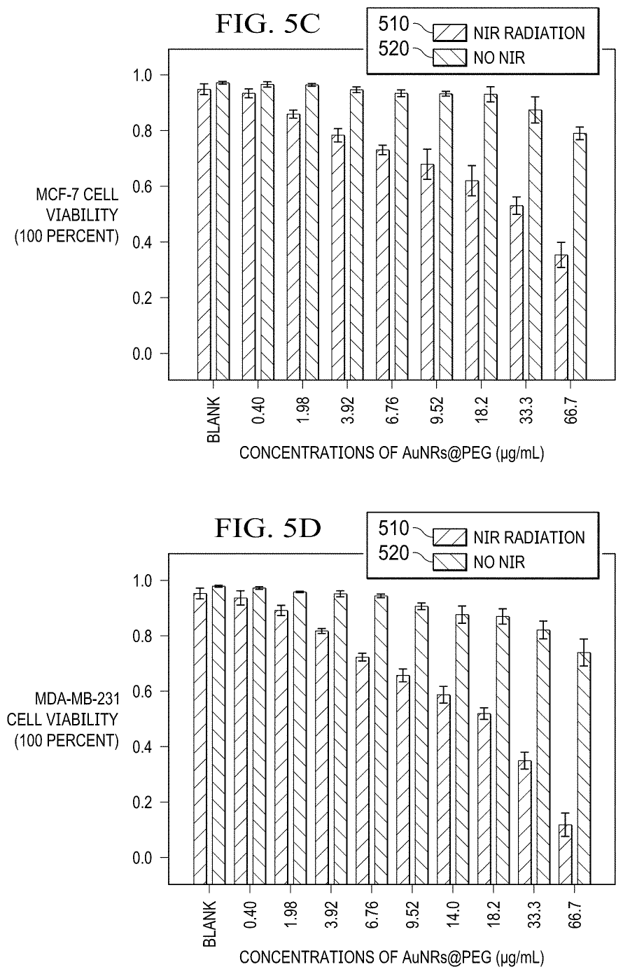

FIGS. 5A-5D are illustrations of in vitro MCF-7 and MDA-MB-231 cell viabilities after incubation with, UCNP@C60 ((A) for MCF-7 and (B) for MDA-MB-231) and AuNRs@PEG @C60 ((C) for MCF-7 and (D) for MDA-MB-231) at different concentrations with 980 nm radiation 510 (left) & without 980 nm radiation 520 (right).

Before the evaluation of therapeutic efficiency of UCNP@C60+AuNRs, separate PDT and PTT were tested (FIG. 5). During these tests, MCF-7 and MDA-MB-231 cells were introduced respectively. As shown in FIG. 5A, after incubating with the UCNP@C60 samples having various concentrations, cell viabilities of MCF-7 were tested by EthD-1&Calcein assays. It is worth mentioning that, without 980 nm laser radiation, the UCNP@C60 samples showed high viabilities of >81%, indicating the excellent biocompatibility of UCNPs@C60. However, when introducing 980 nm radiation, a clear cell viability decrease can be obtained, especially when reaching 37.4 μg/mL, where cell viability decreased to around 93%; while viability without 980 nm radiation, still stayed around 97%. indicating a significant photodynamic effect. When UCNP@C60 concentration reached 275 μg/mL, MCF-7 cell viability significantly decreased to 51%; while without 980 nm radiation, still stayed around 90%. When UCNP@C60 concentration reached 550μg/mL, almost MCF-7 cells have been killed, leaving viability below 5% on average; while without 980 nm radiation, still stayed around 78%. In summary, After UCNP@C60 incubation and then 980 nm light irradiation, a significant number of MCF-7 cells were killed with obviously lower viability than those treated without laser irradiation, resulting from the PDT effect derived from C60, and efficient upconverting from UCNPs. To firmly demonstrate the PDT efficiency, MDA-MB-231 cells were utilized, as shown in FIG. 5B. Similar to MCF-7 cells, various concentrations of UCNP@C60 samples were introduced and incubated, resulting in a similar trend of viability decrease, along with the increase of UCNP@C60 concentrations. When UCNP@C60 concentration reached 550 μg/mL, the MDA-MB-231 cells have been mostly killed, leaving viability below 10% on average; while without 980 nm radiation, still stayed around 75%, indicating a high PDT efficiency of UCNP@C60.

As another important therapeutic factor, PTT efficiency was tested as well. During these tests, MCF-7 and MDA-MB-231 cells were introduced respectively. As shown in FIG. 5C, after incubating with the AuNRs@PEG samples of various concentrations, cell viabilities of MCF-7 were tested by EthD-1&Calcein assays. It is worth mentioning that, without 980 nm laser radiation, samples with AuNRs@PEG showed high viabilities of >79%, indicating the excellent biocompatibility of AuNRs@PEG. However, when introducing 980 nm radiation, a clear cell viability decrease can be obtained, especially when reaching 1.98 μg/mL, where cell viability decreased to around 85%; while viability without 980 nm radiation, still stayed around 95%. indicating a significant photothermal effect. When AuNRs@PEG concentration reached 33.3 μg/mL, MCF-7 cell viability significantly decreased to ˜53%; while without 980 nm radiation, still stayed around 91%. Furthermore, when AuNRs@PEG concentration reached 66.7 μg/mL, more than half the amount of MCF-7 cells have been killed, leaving viability below 35% on average; while without 980 nm radiation, still stayed around 80%. In summary, After AuNRs@PEG incubation and then 980 nm light irradiation, a significant number of MCF-7 cells were killed with obviously lower viability than those treated without laser irradiation, resulting from the PTT effect derived from AuNRs. To firmly demonstrate the PTT efficiency, MDA-MB-231 cells were utilized, as shown in FIG. 5D. Similar to MCF-7 cells, various concentrations of AuNRs@PEG samples were introduced and incubated, resulting in a similar trend of viability decrease, along with the increase of AuNRs@PEG concentrations. When the AuNRs@PEG concentration reached 66.7 μg/mL, the MDA-MB-231 cells were mostly killed, leaving viability around 10% on average; while without 980 nm radiation, still stayed around 75%, indicating a high PTT efficiency of AuNRs@ PEG.

In Vitro Phototherapy Efficiency Tests of UCNP@C60+AuNRs and PDT/PDT Comparison

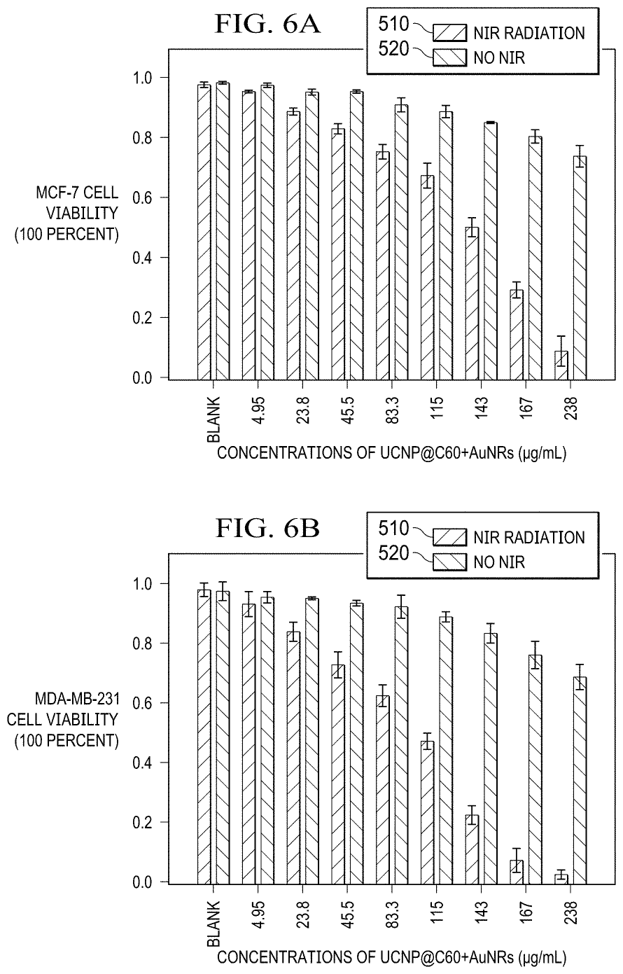

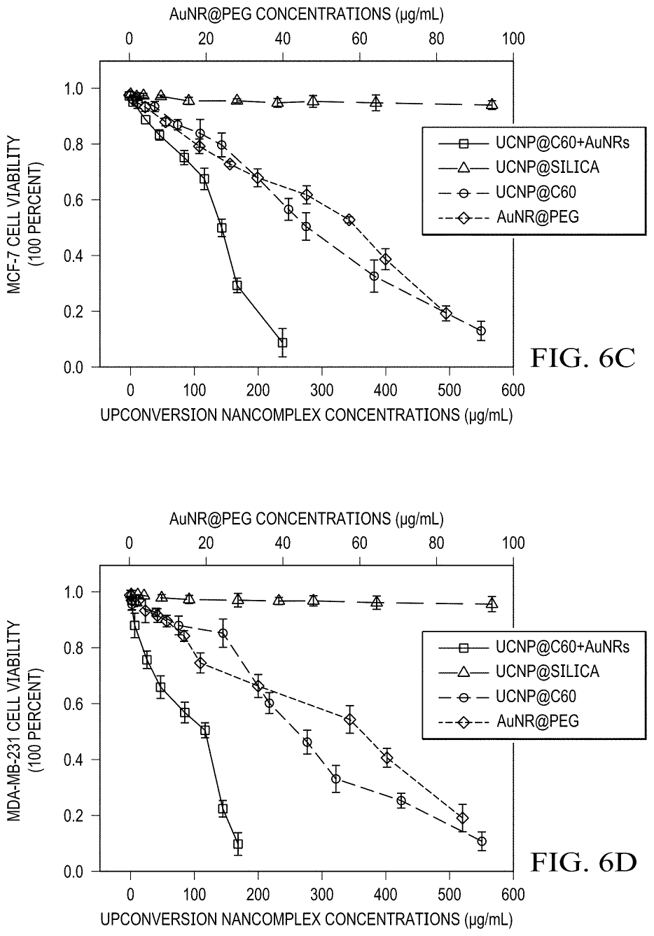

FIGS. 6A-6B show in vitro MCF-7 and MDA-MB-231 cell viabilities after incubation with, UCNP@C60+AuNRs ((A) for MCF-7 and (B) for MDA-MB-231). FIGS. 6C-6D show cell viability comparison with cell viability between PDT (AuNRs@PEG), PDT (UCNPs@C60), and PDT+PTT (UCNPs@C60+AuNRs) ((C) for MCF-7 and (D) for MDA-MB-231).

To evaluate the therapeutic efficiency of UCNP@C60+AuNRs, four groups of MCF-7 and MDA-MB-231 cells (two degrees of malignancy) were treated under different conditions for 24 h, and then cell viability was quantitatively tested using the EthD-1&Calcein assays (FIGS. 6A-6B). In FIG. 6A, after incubating with the UCNP@C60+AuNRs samples of various concentrations, cell viabilities of MCF-7 were tested by EthD-1&Calcein assays. It is worth mentioning that, without 980 nm laser radiation, samples with UCNP@C60+AuNRs showed high viabilities of >75%, indicating the excellent biocompatibility of UCNP@C60+AuNRs. However, when introducing 980 nm radiation, a clear cell viability decrease can be obtained, especially when reaching 23.8 μg/mL, where cell viability decreased to around 88%; while viability without 980 nm radiation, still stayed around 94%. indicating a significant phototherapeutic efficiency. When UCNP@C60+AuNRs concentration reached 143 μg/mL, MCF-7 cell viability significantly decreased to ˜49%; while without 980 nm radiation, still stayed around 86%. Furthermore, when UCNP@C60+AuNRs concentration reached 238 μg/mL, MCF-7 cells have been mostly killed, leaving viability below 9% on average; while without 980 nm radiation, still stayed around 74%. In summary, After A UCNP@C60+AuNRs incubation and then 980 nm light irradiation, a significant number of MCF-7 cells were killed with obviously lower viability than those treated without laser irradiation, resulting from a combined PTT+PDT effects derived from AuNRs@PEG and UCNP@C60 respectively. To firmly demonstrate the PTT+PDT efficiency, MDA-MB-231 cells were utilized, as shown in FIG. 6B. Similar to MCF-7 cells, various concentrations of UCNP@C60+AuNRs samples were introduced and incubated, resulting in a similar trend of viability decrease, along with the increase of UCNP@C60+AuNRs concentrations. When the UCNP@C60+AuNRs concentration reached 167 μg/mL, the MDA-MB-231 cells were mostly killed, leaving viability around 7% on average; while without 980 nm radiation, still stayed around 70%, indicating a high PTT+PDT therapeutic efficiency of UCNP@C60+AuNRs.

Thereafter, single PDT, PTT, and combined PDT+PTT were together plotted to verify the assumption of synergistic therapeutic effect. Comparing the viabilities, with UCNP@C60 alone, even under the same concentrations, UCNP@C60+AuNRs would generate simultaneous PDT and PTT, resulting in a significant decrease in cell viabilities from 24 h incubation of MCF-7 cells (FIG. 6C). It is worth mentioning that, in FIG. 6 C&D, the concentrations of AuNRs@PEG were longitudinally corresponding to the same amount of the AuNRs portion from UCNP@C60+AuNRs. When cells were treated with UCNP@C60+AuNRs samples and then NIR light irradiation, the viabilities were obviously decreased comparing samples with UCNP@C60 and AuNRs@PEG alone from the NIR laser-treated groups. In detail, at the same conditions, the viability of MCF-7 reached below 10% at 238 μg/mL of UCNP@C60+AuNRs, while 550 μg/mL of UCNP@C60. We assume this interesting finding should be due to the synergistic effect between PDT and PTT. In general, during laser radiation, UCNPs will transfer 980 nm photons into visible light, and sequentially C60 gets excited and generate ROS from the surrounding environment to reach PDT. Meanwhile, AuNRs also get excited from 980 nm radiation, generating heat to the microenvironment to reach PTT. We assume that the temperature increase from PTT will enhance the ROS generation efficiency, resulting in a higher PDT efficiency comparing PDT without heating. After calculations, we found that, for MCF-7 cells, the IC₅₀ of UCNP@C60+AuNRs is 135 μg/mL, which is 52% decreased comparing the IC₅₀ of UCNP@C60 (280 μg/mL). The UCNP@C60+AuNRs and NIR-treated group have the highest cell killing efficacy, which demonstrates that this upconverting nanocomplex makes the best utilization of the 980 nm radiation. To further demonstrate the synergistic effect between PTT and PDT, viabilities of MDA-MB-231 cells were plotted together as well. In the image of FIG. 6D, at the same conditions, the viability of MDA-MB-231 reached below 10% at 167 μg/mL of UCNP@C60+AuNRs, while 550 μg/mL of UCNP@C60. After calculations, we found that, for MDA-MB-231 cells, the IC₅₀ of UCNP@C60+AuNRs is 115 μg/mL, which is 55% decreased comparing the IC₅₀ of UCNP@C60 (255 μg/mL). Obviously, from viability data of both MDA-MB-231 and MCF-7 cells, the UCNP@C60 and UCNP@C60+AuNRs with 980 nm laser-irradiated groups have lower cell-killing efficacy than that of the UCNP@C60 and AuNRs@PEG separately. In summary, from the data comparison and viability differences, the synergistic effects between PDT and PTT have been revealed and verified.

Phototherapy Efficiency Tests Via In Vivo Toxicity

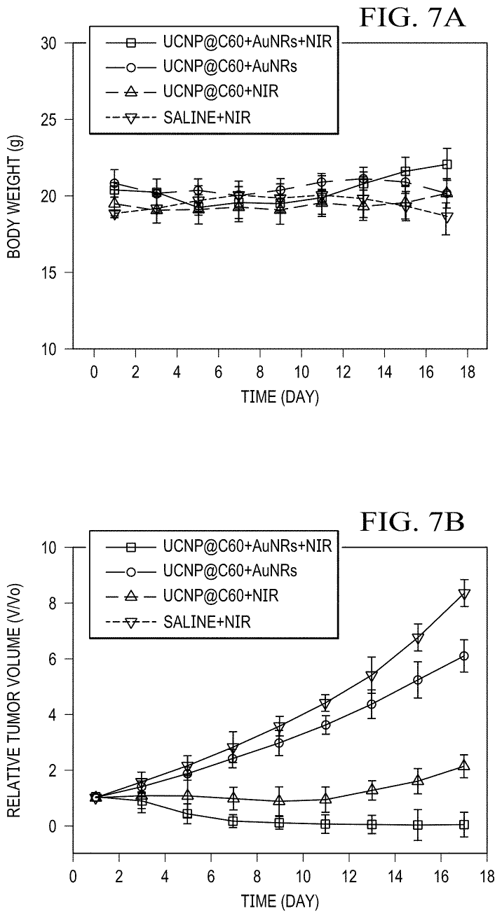

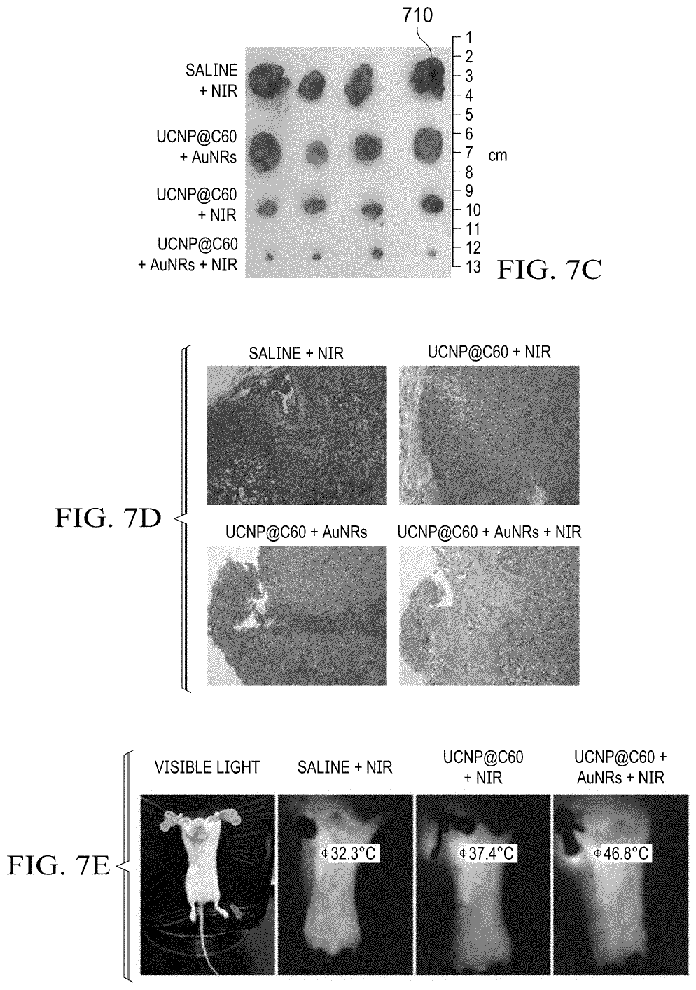

FIG. 7A shows changes in the body weights. FIG. 7B shows relative tumor volume achieved from mice with varying treatments. FIG. 7C shows photographs of excised tumors from representative mice. FIG. 7D shows H&E stained images of tumor tissues 710 obtained after 14 days of treatment. FIG. 7E shows thermal imaging with/without NIR radiation with varying treatments

To further verify the therapeutic effect of our design, in vivo tests were investigated. The 4T1 cells were injected subcutaneously in the left axilla of each mouse (about 20 g) to obtain tumors. When the tumors grew to 6-8 mm (60-100 mm³), the mice were randomly divided into 4 groups (6 per group) and injected in vein with 0.2 mL: (1) saline as blank; (2) UCNP@C₆₀+AuNRs (20 mg/kg in 0.2 mL PBS) as for PTT+PDT; (3) UCNP@C₆₀+AuNRs (20 mg/kg in 0.2 mL PBS) as for NIR control; and (4) UCNP@C₆₀ (20 mg/kg in 0.2 mL PBS) as for PDT alone, respectively. In addition, the 980 nm laser irradiation of tumor sites was executed for the group (1), (2), and (4) after the injection. The body weight and the tumor size were recorded every 2 days after the initial treatments. FIGS. 7A-7B exhibit the average body weights and the average tumor volume of each group during the 17 days treatments. As presented in FIG. 7A, there is no significant body weight decrease or increase in these 4 groups, indicating that the samples have no adverse drug reaction to the mice. Compared with the control group, the growth of tumors on NIR light irradiated mice is slightly inhibited over 2 weeks of treatment, which could be caused by the heat effect originating from the hemoglobin absorbance. Meanwhile, in FIG. 7B the mice injected with the UCNP@C₆₀+AuNRs and exposed to the NIR light show a much smaller tumor size and much lower relative tumor volume (RTV) than the other NIR-irradiated groups, which mainly originate from the combined PDT and PTT effects of UCNP@C₆₀+AuNRs. The tumor growth on UCNP@C60 and NIR-treated mice are slightly inhibited to a certain degree (RTV from 1.0 to 0.84) at the first week, which may be ascribed to the PDT effect of UCNP@C₆₀. The UCNP@C₆₀+AuNRs sample has higher anticancer efficacy than UCNP@C60, mainly due to the synergistic effect of PTT from AuNRs. It is noted that the mice treated with UCNP@C₆₀+AuNRs injection and NIR irradiation display tumor volume even smaller than the initial size (RTV from 1.0 to 0.022). Moreover, during the investigation period, the bodyweight of five groups was not obviously affected, which demonstrates an ignorable side effect of the designed upconversion nano complex to the mice. It is worth mentioning that, as a comparison, the mice injected with the UCNP@C60+AuNRs without the NIR light show a much bigger tumor size and much higher relative tumor volume (RTV) than the others with NIR-irradiated groups, which indicated that NIR played a significant role in originating the upconverting system and triggering the phototherapy nanosystem. In FIG. 7C, the digital photographs of tumors collected from representative mice also confirm that the tumor volume upon UCSM and NIR treatment is the smallest, revealing its highest tumor inhibition efficiency. As further proof, hematoxylin and eosin (H&E) stained tumor sections displayed in FIG. 7D show the highest level of tissue damage for the group injected with the UCNP@C60+AuNRs sample and irradiated with a NIR laser, illustrating good consistency with the data of tumor growth. In FIG. 7E, the temperature increases were captured with an IR camera as well. During the 5 min 980 nm radiation, mice with the UCNP@C60+AuNRs sample showed 46.8° C., with more than 25° C. increase, indicating the high PTT efficiency from the conjugated AuNRs. It is noted that the mice treated with saline injection and NIR irradiation also displayed temperature increases, mainly because of the water absorption at 980 nm (slight PTT), which also provides a reason for the relative lower RTV of saline+NIR than UCNP@C60+AuNRs (without NIR). All in all, the developed UCNP@C60+AuNRs holds great promise as a biocompatible nanomedicine for simultaneous PDT+PTT clinical applications.

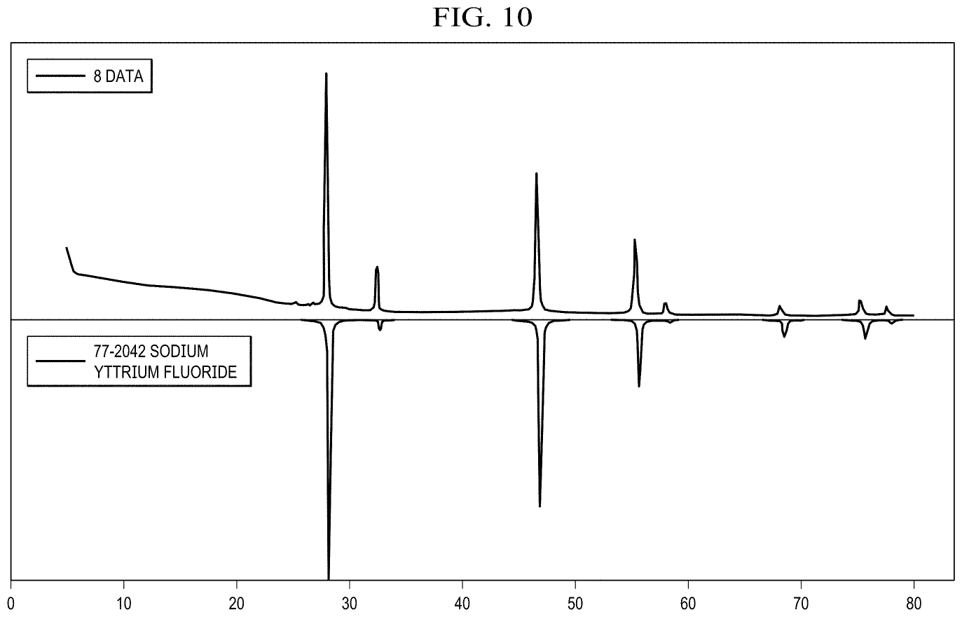

FIG. 10 shows X-ray diffraction analysis of the UCNPs compared with standard data of cubic phase NaYF4,Yb,Er (JCPDS No. 77-2042). FIG. 10 confirms that embodiments of this disclosure included cubic phase NaYF4,Yb,Er (JCPDS No. 77-2042).

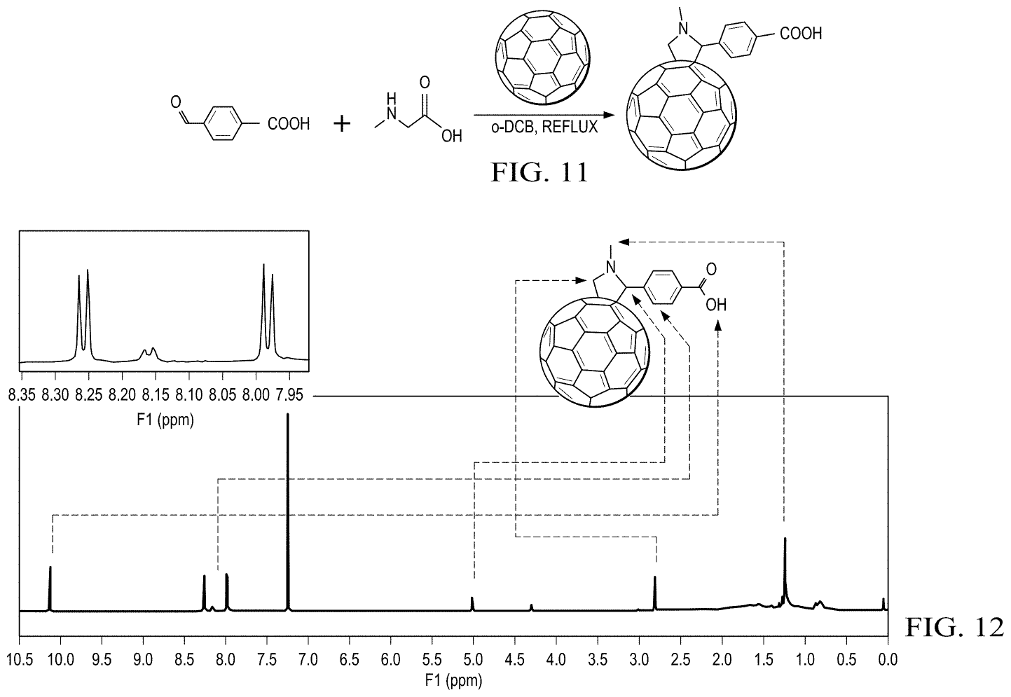

FIG. 11 shows the carboxyl functionalized Fullerene derivative was synthesized through a typical Prato reaction. Briefly, a mixture of 4-carboxybenzaldehyde (0.210 g, 1.40 mmol), C60 (0.202 g, 0.28 mmol), and N-methylglycine (0.125 g, 1.40 mmol) in chlorobenzene (60 mL) was refluxed overnight under a nitrogen atmosphere. The solvent was removed by rotary evaporation under reduced pressure. The crude product was purified over silica gel column chromatography with toluene to toluene/THF (2/1) as the eluents to afford a brown-yellow solid (92.7 mg, 37%).

FIG. 12 shows ¹H-NMR characterization of the synthesized Carboxyl C60. FIG. 12 confirms ¹H NMR (300 MHz, DMSO-d₆): δ 2.20 (s, 3H), 6.65 (s, 1H), 6.89 (s, 2H), 8.03 (d, J) 8.4 Hz, 2H), 8.15 (d, J) 8.4 Hz, 2H), 10.12 (s, 1H). Calculated for C₇₀H₁₁NO₂: C, 93.64; H, 1.23; N, 1.56. Found: C, 93.45; H, 1.31; N, 1.62. ESI-MS (m/z): calculated, 897.1; found, 897.0.

FIG. 13 shows MALDI-TOF characterization for synthesized Carboxyl C60. FIG. 13 confirms that embodiments of this disclosure included synthesized carboxyl C60.

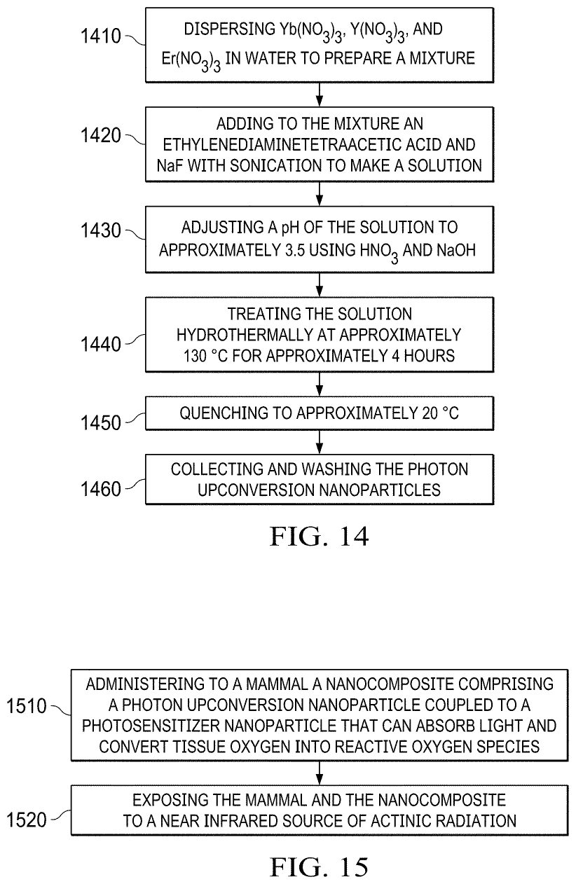

FIG. 14 shows a sub-generic low temperature hydrothermal method of making photon upconversion nanoparticles. A preferred embodiment of this disclosure comprises dispersing 1410 Yb(NO₃)₃, Y(NO₃)₃, and Er(NO₃)₃ in water to prepare a mixture. A preferred embodiment of this disclosure comprises adding 1420 to the mixture an ethylenediaminetetraacetic acid and NaF with sonication to make a solution. A preferred embodiment of this disclosure comprises adjusting 1430 a pH of the solution to approximately 3.5 using HNO₃ and NaOH. A preferred embodiment of this disclosure comprises treating 1440 the solution hydrothermally at approximately 130° C. for approximately 4 hours. A preferred embodiment of this disclosure comprises quenching 1450 to approximately 20° C. A preferred embodiment of this disclosure comprises 1460 collecting and washing the photon upconversion nanoparticles.

Optionally, dispersing further comprises stirring and ultrasonic agitation. Optionally, adding further comprises sonicating. Optionally, embodiments of the method can also comprise modifying a surface of the photon upconversion nanoparticles comprising silanization using ammonium hydroxide and tetraethyl orthosilicate. Optionally, modifying further comprises amine terminal decoration using aminopropyltrimethoxysilane. Optionally, embodiments of the method can also comprise reacting the photon upconversion nanoparticles with a benzoyloxy pyrrolidine based C60 derivative using carbodiimide coupling.

FIG. 15 shows a sub-generic near infrared triggered photon upconversion method for synergistic photodynamic and photothermal cancer therapy of a mammal in need thereof. A preferred embodiment of this disclosure comprises administering 1510 to the mammal a nanocomposite comprising a photon upconversion nanoparticle coupled to a photosensitizer nanoparticle that can absorb light and convert tissue oxygen into reactive oxygen species. A preferred embodiment of this disclosure comprises exposing 1520 the mammal and the nanocomposite to a near infrared source of actinic radiation.

Optionally, the photon upconversion nanoparticle comprises NaYF₄:Yb/Er. Optionally, the photon upconversion nanoparticle comprises silanization moieties. Optionally, the photon upconversion nanoparticle comprises terminal amines. Optionally, the photosensitizer nanoparticle comprises a benzoyloxy pyrrolidine based C60 derivative. Optionally, the benzoyloxy pyrrolidine based C60 derivative is carbodiminde coupling grafted to the photon upconversion nanoparticle. Optionally, the nanocomposite also comprises gold nanorods covalently conjugated with the photon upconversion nanoparticle. Optionally, the gold nanorods are carboxylic polyethylene glycol functionalized.

### CONCLUSIONS

In summary, we utilized 980 nm sensitized NaYF₄:Yb/Er nanocrystal, covalently conjugating C60 AuNRs for a simultaneous PTT and PDT treatment. The NaYF₄:Yb/Er nanocrystal with optimal 980 nm coverage and can efficiently transduce the 980 nm photons to green and red light. The carboxylic-modified C60 and PEG-coated AuNRs were covalently conjugated onto the silica shell of the UCNPs. The spectral overlaps between the maximum absorption of AuNRs and 980 nm radiation, along with between upconverted visible emissions and C60, take full advantages of the high PTT efficiency and PDT activation to generate cytotoxic ROS for simultaneous antitumor therapy. The therapeutic evaluation of product UCNP@C60+AuNRs was successfully validated both in vitro (breast cancer cell lines MCF-7 and MDA-MB-231) and in vivo (Mouse 4T1 breast tumor model), exhibiting significant PDT+PDT synergistic effects in cancer therapy (e.g., 53% higher than PDT alone). Moreover, the UCNP@C60+AuNRs showed the synergistic effects between PDT and PTT under a single 980 nm light excitation, revealing its potency in the tumor therapeutic field.

The different illustrative examples describe components that perform actions or operations. In an illustrative embodiment, a component can be configured to perform the action or operation described. For example, the component can have a configuration or design for a structure that provides the component an ability to perform the action or operation that is described in the illustrative examples as being performed by the component. Further, To the extent that terms “includes”, “including”, “has”, “contains”, and variants thereof are used herein, such terms are intended to be inclusive in a manner similar to the term “comprises” as an open transition word without precluding any additional or other elements.

The descriptions of the various embodiments of the present invention have been presented for purposes of illustration, but are not intended to be exhaustive or limited to the embodiments in the form disclosed. Not all embodiments will include all of the features described in the illustrative examples. Further, different illustrative embodiments may provide different features as compared to other illustrative embodiments. Many modifications and variations will be apparent to those of ordinary skill in the art without departing from the scope and spirit of the described embodiment. The terminology used herein was chosen to best explain the principles of the embodiment, the practical application or technical improvement over technologies found in the marketplace, or to enable others of ordinary skill in the art to understand the embodiments disclosed here.

What is claimed is:

1. A composition of matter, comprising: a nanocomposite comprising a photon upconversion nanoparticle coupled to a photosensitizer nanoparticle that can absorb light and convert tissue oxygen into reactive oxygen species.

2. The composition of matter of claim 1, wherein the photon upconversion nanoparticle comprises NaYF₄:Yb/Er.

3. The composition of matter of claim 2, wherein the photon upconversion nanoparticle comprises silanization moieties.

4. The composition of matter of claim 3, wherein the photon upconversion nanoparticle comprises terminal amines.

5. The composition of matter of claim 4, wherein the photosensitizer nanoparticle comprises a benzoyloxy pyrrolidine based C₆₀ derivative.

6. The composition of matter of claim 5, wherein the benzoyloxy pyrrolidine based C₆₀ derivative is carbodiminde coupling grafted to the photon upconversion nanoparticle.

7. The composition of matter of claim 1, further comprising a photo thermal nanomaterial that converts light to heat for cancer therapy, the photothermal nanomaterial comprising gold nanorods or other photothermal nanoparticles, covalently conjugated with the photon upconversion nanoparticle.

8. The composition of matter of claim 7, wherein the gold nanorods are carboxylic polyethylene glycol functionalized.

9. A low temperature hydrothermal method of making photon upconversion nanoparticles, the method comprising:
dispersing Yb(NO₃)₃, Y(NO₃)₃, and Er(NO₃)₃ in water to prepare a mixture; then
adding to the mixture an ethylenediaminetetraacetic acid and NaF with sonication to make a solution; then
adjusting a pH of the solution to approximately 3.5 using HNO₃ and NaOH; then
treating the solution hydrothermally at approximately 130° C. for approximately 4 hours; then
quenching to approximately 20° C.; and then
collecting and washing the photon upconversion nanoparticles.

10. The low temperature hydrothermal method of claim 9, wherein dispersing further comprises stirring and ultrasonic agitation.

11. The low temperature hydrothermal method of claim 9, wherein adding further comprises sonicating.

12. The low temperature hydrothermal method of claim 9, further comprising modifying a surface of the photon upconversion nanoparticles comprising silanization using ammonium hydroxide and tetraethyl orthosilicate.

13. The low temperature hydrothermal method of claim 12, wherein modifying further comprises amine terminal decoration using aminopropyltrimethoxysilane.

14. The low temperature hydrothermal method of claim 13, further comprising reacting the photon upconversion nanoparticles with a benzoyloxy pyrrolidine based C₆₀ derivative using carbodiimide coupling.

15. A near infrared triggered photon upconversion method for synergistic photodynamic and photothermal cancer therapy of a mammal in need thereof, the method comprising:
administering to the mammal a nanocomposite comprising a photon upconversion nanoparticle coupled to a photosensitizer nanoparticle that can absorb light and convert tissue oxygen into reactive oxygen species and a photothermal nanomaterial that converts light to heat; and
exposing the mammal and the nanocomposite to a near infrared source of actinic radiation.

16. The near infrared triggered photon upconversion method of claim 15, wherein the photon upconversion nanoparticle comprises NaYF₄:Yb/Er.

17. The near infrared triggered photon upconversion method of claim 16, wherein the photon upconversion nanoparticle comprises silanization moieties.

18. The near infrared triggered photon upconversion method of claim 17, wherein the photon upconversion nanoparticle comprises terminal amines.

19. The near infrared triggered photon upconversion method of claim 18, wherein the photosensitizer nanoparticle comprises a benzoyloxy pyrrolidine based C₆₀ derivative.

20. The near infrared triggered photon upconversion method of claim 19, wherein the benzoyloxy pyrrolidine based C₆₀ derivative is carbodiminde coupling grafted to the photon upconversion nanoparticle.

21. The near infrared triggered photon upconversion method of claim 15, wherein the photothermal nanomaterial comprises gold nanorods or other photothermal nanoparticles, covalently conjugated with the photon upconversion nanoparticle.

22. The near infrared triggered photon upconversion method of claim 21, wherein the gold nanorods are carboxylic polyethylene glycol functionalized.
